(19) United States
(12) Patent Application Publication
(10) Pub. No.: US 2011/0111017 A1
(43) Pub. Date: May 12, 2011

(54) USE OF IMMUNOGENIC COMPOSITIONS FOR THE TREATMENT OR PREVENTION OF PATHOGEN INFECTIONS

(75) Inventors: Catharine M. Bosio, Hamilton, MT (US); John T. Belisle, Fort Collins, CO (US); Jeffrey Fairman, Burlingame, CA (US)

(73) Assignee: GOVERNMENT OF THE UNITED STATES OF AMERICA,AS REP. BY THE SECRETARY,DEPT. OF HEALTH AND HUMAN SERVIC, Rockville, MD (US)

(21) Appl. No.: 12/919,204

(22) PCT Filed: Feb. 24, 2009
(86) PCT No.: PCT/US09/01176

§ 371 Date: Nov. 23, 2010

Related U.S. Application Data
(60) Provisional application No. 61/030,984, filed on Feb. 24, 2008.

(51) Int. Cl. A61K 9/127; A61K 39/145; A61K 39/02; A61K 39/09; A61K 39/095; A61K 39/00; A61P 37/00

(52) U.S. Cl. 424/450; 424/209.1; 424/234.1; 424/244.1; 424/250.1; 424/265.1; 424/274.1

(57) ABSTRACT

The invention provides compositions featuring cationic liposome nucleic acid molecules complexed with pathogen derived antigen and methods of using such compositions for the treatment or prevention of an infectious disease. Other features and advantages of the invention will be apparent from the detailed description, and from the claims.

### CROSS-REFERENCE TO RELATED APPLICATION

This application claims the benefit of the following U.S. Provisional Application No. 61/030,984, filed on Feb. 24, 2008, the entire contents of which are incorporated herein by reference.

### STATEMENT OF RIGHTS TO INVENTIONS MADE UNDER FEDERALLY SPONSORED RESEARCH

This work was supported by the National Institutes of Health. The government has certain rights in the invention.

### BACKGROUND OF THE INVENTION

Antibiotics have been widely used to treat a wide variety of diseases (e.g., pneumonia, tuberculosis) and to prevent skin wounds from becoming infected. While the use of antibiotics has saved the lives of patients who a century ago would have certainly died from infection, the wide-spread use of antibiotics for medical and agricultural purposes has caused an increase in antibiotic-resistant bacteria, such as methicillin-resistant Staphylococcus aureus, vancomycin-resistant enterococcus, and extreme drug-resistant tuberculosis. In the United States and globally, many infectious pathogens, including those that cause pneumonia, ear infections, acne, gonorrhea, urinary tract infections, meningitis, and tuberculosis, have developed some level of resistance to commonly used antibiotics and antimicrobials. Drug resistance to conventional antibiotics has likely contributed to a dramatic increase in infectious disease deaths. Compositions that are effective against antibiotic-resistant pathogens are urgently required.

Even in the absence of antibiotic resistance, conventional antibiotic therapies are not universally effective against all pathogens. For example, Francisella tularensis is the causative agent of an acute, lethal, bacterial disease, Tularemia. F. tularensis infections are susceptible to antibiotic therapy only where the therapy is initiated within forty-eight hours of infection. At this early stage of infection, many patients have not yet been identified as having an F. tularensis infection. Even where appropriate antibiotic therapy is rapidly initiated, antibiotic therapy may be incomplete and relapse of the disease can occur. Further complicating effective treatment is the fact that wild-type Francisella can develop resistance to commonly used antibiotics. Currently available methods of preventing tularemia are also inadequate. As little as 10 CFU of F. tularensis is sufficient to cause a lethal infection in humans and other mammals, and the only existing vaccine fails to provide long lasting protection against aerosols greater than 100 CFUs.

Accordingly, improved methods of treating or preventing infectious diseases are urgently required.

### SUMMARY OF THE INVENTION

As described below, the present invention features immunogenic compositions and uses thereof for the treatment or prevention of a pathogen infection.

In one aspect, the invention generally provides a vaccine containing or consisting essentially of a liposome, a non-coding nucleic acid molecule, and a bacterial, fungal, or parasitic antigen in a pharmaceutically acceptable excipient, where the vaccine induces a protective immune response in a vaccinated mammal (e.g., human).

In another aspect, the invention provides a vaccine for inducing an F. tularensis specific immune response in a subject, the vaccine containing or consisting essentially of a complex containing an effective amount of a cationic liposome, a non-coding nucleic acid molecule, and a bacterial membrane fraction in a pharmaceutically acceptable excipient.

In a related aspect, the invention provides a method of immunizing a mammal containing administering to the mammal a vaccine containing an effective amount of a liposome, a non-coding nucleic acid molecule, and a bacterial antigen in a pharmaceutically acceptable excipient.

In another related aspect, the invention provides a method of treating or preventing a pathogen infection in a mammal in need thereof involving administering to the mammal an effective amount of a composition containing a liposome, a non-coding DNA, and a bacterial antigen in a pharmaceutically acceptable excipient.

In yet another aspect, the invention provides a method for inducing an F. tularensis specific immune response, the method involving administering to a mammal an effective amount of a composition containing cationic liposome, a non-coding nucleic acid molecule, and a bacterial membrane fraction in a pharmaceutically acceptable excipient.

In yet another aspect, the invention provides a kit for preventing or treating a pathogen infection, the kit containing an effective amount of a cationic liposome, a non-coding nucleic acid molecule, and a pathogen antigen (e.g., bacterial, viral, or fungal antigen present in a membrane fraction). In one embodiment, the cationic liposome and non-coding nucleic acid molecule are present in a cationic liposome DNA complex. In another embodiment, the kit contains written instructions.

In various embodiments of the above aspects or of other aspects delineated herein, the composition is administered intraperitoneally (IP), subcutaneously (SC) or intranasally. In other embodiments of the above aspects, the composition induces production of proinflammatory cytokines from antigen presenting cells, reduces intracellular pathogen replication, or increases survival of the mammal following pathogen exposure compared to an untreated control mammal. In other embodiments of the above aspects the composition generates pathogen specific IgM in the mammal. In other embodiments, the pathogen antigen (e.g., Burkholderia pseudomallei, Yersinia pestis. and Influenza virus, fungal, or parasitic antigen) is provided in a membrane fraction. In still other embodiments, the liposome is a cationic liposome. In still other embodiments, the vaccine prevents or treats a bacterial, fungal, or parasitic infection of the mammal. In yet other embodiments, the bacteria is a gram positive or gram negative bacteria (e.g., Francisella tularensis). In yet other embodiments of the above aspects, the antigen and the nucleic acid molecule are complexed to or within the liposome. In still other embodiments of the above aspects, the bacterial, fungal, or parasitic antigen is provided in a membrane fraction. In still other embodiments of any invention delineated herein, the composition or method is useful for the treatment or prevention of bacterial meningitis, tularemia, influenza, plague, mellidosis, and pneumonicoccal mediated disease.

The invention provides compositions featuring cationic liposome DNA complexed with pathogen derived antigen and methods of using such compositions for the treatment or prevention of an infectious disease. Other features and advantages of the invention will be apparent from the detailed description, and from the claims.

### DEFINITIONS

By “ameliorate” is meant decrease, suppress, attenuate, diminish, arrest, or stabilize the development or progression of a disease.

By “antigen” is meant an agent that induces a humoral and/or cellular immune response.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “cationic liposome DNA complex (CLDC)” is meant a complex comprising cationic liposomes in association with non-coding DNA polynucleotides. CLDC's are known in the art and are described, for example, by Canonico et al., 1994. No lung toxicity after repeated aerosol or i.v. delivery of plasmid cationic liposome complexes. J. Appl. Physiol. 77:415, and by

By “complex” is meant physically associate. Association in a complex can be mediated, for example, by attractions between molecules of different charge, or by hydrophobic or hydrophilic interactions.

By “disease” is meant any condition or disorder that damages or interferes with the normal function of a cell, tissue, or organ. Examples of diseases include bacterial invasion or colonization of a host cell.

By “effective amount” is meant the amount of a required to ameliorate the symptoms of a disease relative to an untreated patient. The effective amount of active compound(s) used to practice the present invention for therapeutic treatment of a neurodegenerative disease varies depending upon the manner of administration, the age, body weight, and general health of the subject. Ultimately, the attending physician or veterinarian will decide the appropriate amount and dosage regimen. Such amount is referred to as an “effective” amount.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA.

By “liposome” is meant a microscopic vesicle comprising an aqueous core enclosed in one or more phospholipid layers.

By “pathogen” is meant any bacteria, viruses, fungi, or protozoans capable of interfering with the normal function of a cell.

Exemplary bacterial pathogens include, but are not limited to, Aerobacter, Aeromonas, Acinetobacter, Agrobacterium, Bacillus, Bacteroides, Bartonella, Bordtella, Brucella, Burkholderia, Calymmatobacterium, Campylobacter, Citrobacter, Clostridium, Cornyebacterium, Enterobacter, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia.

By “protective immune response” is meant an immune response sufficient to ameliorate a pathogen infection in a mammal.

By “reference” is meant a standard or control condition.

By “subject” is meant a mammal, including, but not limited to, a human or non-human mammal, such as a bovine, equine, canine, ovine, or feline.

By “vaccine” is meant an immunogenic composition.

### BRIEF DESCRIPTION OF THE DRAWINGS

FIG. 1 provides a schematic diagram of the experimental protocol used to generate an F. tularensis specific immune response in vitro and in vivo.

FIG. 2 is a graph showing that cationic liposome DNA complex (CLDC) alone protected against intranasal infections of F. tularensis live vaccine strain (LVS). Balb/c mice (n=5/group) were injected intraperitoneally with CLDC three days prior to exposure to a lethal intranasal challenge of F. tularensis LVS. Survival was monitored over time. All mice treated with CLDC survived challenge, whereas all untreated mice succumbed to infection 5 days after exposure.

FIG. 3 is a graph showing that Balb/c mice (n=10/group) were injected with CLDC intraperitoneally (IP), subcutaneously (SC) or intranasally (TN) three days prior to aerosol challenge with F. tularensis Schu4. Mice treated with CLDC alone (i.e., in the absence of F. tularensis antigens) did not exhibit significantly increased survival compared to untreated mice. One out of ten mice treated with CLDC SC survived Schu4 challenge.

FIG. 4 provides four panels showing that CLDC and Francisella antigens induced production of proinflammatory cytokines from antigen presenting cells. CLDC and live vaccine strain (LVS) membrane fraction proteins (MPF) (1 ug/ml) were added to bone marrow derived macrophages or dendritic cells (BMWØ and BMDC, respectively) obtained from Balb/c mice. Cultures were incubated overnight. Supernatants were collected and assessed for various cytokines by ELISA. CLDC+MPF in combination enhanced production of IFN-β in BMW) and BMDC compared to cells treated with CLDC alone (*=p<0.05). CLDC+MPF treated BMDC also produced significantly more IL-12/IL-23p40 and IL-6 compared to CLDC or MPF treated BMDC (*=p<0.05). In contrast, CLDC+MPF significantly reduced the concentration of TNF-α secreted by BMDC and IL-12/IL-23p40 from BMMØ compared to either cell type treated with either CLDC or MPF alone (**=p<0.05). Bars represent SEM. Data is representative of three experiments of similar design.

FIG. 5 provides two graphs showing that CLDC+Francisella antigens reduce intracellular replication in vitro. BMMØ and BMDC were treated with CLDC, LVS MPF or CLDC+LVS MPF 24 hours prior to infection with F. tularensis Schu4. Cells were infected with an MOI=5 of F. tularensis Schu4, treated with gentamicin, washed extensively and monitored for intracellular infection and replication of Schu4 at the indicated time points. FIG. 5A shows that pretreatment of BMMØ with CLDC, MPF or CLDC+MPF significantly increased uptake of Schu4 compared to untreated cells immediately after infection (*=p<0.01). Only BMMØ treated with CLDC+MPF controlled replication of Schu4 over time compared to CLDC, MPF or untreated cells (**=p<0.001). Twenty-four hours after infection, BMW) treated with CLDC MPF alone had significantly higher numbers of intracellular Schu4 compared to untreated cells (***=p<0.001). FIG. 5B shows that pretreatment of BMDC with CLDC+MPF significantly inhibited uptake of Schu4 immediately after infection compared with untreated cells (*=p<0.05). In contrast to BMMØ, BMDC treated with either CLDC or CLDC+MPF significantly controlled replication of Schu4 compared to untreated controls or cells treated with MPF alone (**=p<0.01). Lines represent the limit of detection. Bars represent SEM. Data is representative of two experiments of similar design.

FIGS. 6A-6C are graphs showing that CLDC in combination with Francisella antigens elicited rapid protective immunity against F. tularensis Schu4. Mice (n=10/group) were treated with either CLDC or LVS MPF (10 ug/mouse) intraperitoneally (IP) or CLDC+LVS MPF intravenously (IV), subcutaneously (SC) or IP three days prior to aerosol challenge with F. tularensis Schu4. Untreated mice served as negative controls. Survival was monitored over time. FIG. 6A shows that pretreatment of mice with CLDC or MPF alone did not significantly increase survival or mean time to death compared to untreated controls. FIG. 6B shows that CLDC+MPF significantly increased survival following Schu4 infection compared to untreated mice. IP administration of CLDC+MPF offered the greatest protection with 80% survival compared to 30% survival among SC treated mice and 20% survival among mice receiving CLDC+MPF IV. FIG. 6C shows that pretreatment of mice with CLDC+MPF, either SC or IP, significantly increased mean time to death compared to mice given CLDC+MPF IV or untreated controls (*=p<0.05). Bars represent SEM. Data is representative of two experiments of similar design.

FIGS. 7A and 7B show that the number of plasma cells increased in CLDC treated mice. Mice (n=2/group) were injected intraperitoneally with 5% dextrose water (control), LVS MPF (10 ug/mouse), CLDC or CLDC+LVS MPF (10 ug/mouse). Three days later the indicated tissues and/or cells were collected and assessed for cellular content and activation by flow cytometry. FIG. 7A shows the results of FACS analysis showing that CLDC treatment with or without MPF increased the percentage of plasma cells (B220+/CD19+/Ly6C+) in the spleens and mediastinal lymph nodes (MLN) compared to controls or MPF treated mice. In contrast, the percentage of plasma cells in the lungs was relatively unchanged among each group tested. Furthermore, the percentage of B cells and plasma cells in the peritoneum (PEC) were reduced in CLDC and CLDC+MPF treated mice compared to controls and mice injected with MPF alone. FIG. 7B shows that CLDC and CLDC+MPF significantly increased the numbers of plasma cells, but not total B cells, in the spleen compared to controls or mice treated with MPF alone (*=p<0.01). Bars represent SEM. Data is representative of two experiments of similar design.

FIGS. 8A and 8B show that mice treated with Francisella antigens generated specific IgM within 3 days of treatment. Mice (n=2/group) were injected intraperitoneally with 5% dextrose water (control), MPF (10 ug/mouse), CLDC or CLDC+MPF (10 ug/mouse). Three days later serum was collected and assessed for IgM against LVS MPF via western blot (FIG. 8A) or IgM directed against whole F. tularensis Schu4 by ELISA (FIG. 8B). FIG. 8A shows that MPF in the presence or absence of CLDC elicited production of MPF specific serum IgM. FIG. 8B shows that mice that received MPF (with or without CLDC) generated IgM that recognized intact F. tularensis Schu4. CLDC increased the amount of Schu4 specific IgM in CLDC+MPF treated mice. Data is representative of two experiments of similar design.

FIG. 9 is a schematic diagram showing the effect of CLDC and MPF alone and in combination.

FIGS. 10A-10C show that CLDC+MPF controls replication of F. tularensis in mouse macrophages. Mouse bone marrow derived macrophages were treated with 5% Dextrose water (untreated), LVS membrane protein fraction (MPF), cationic liposome DNA complexes (CLDC), or CLDC+MPF overnight. Cells were then infected with F. tularensis Schu4. At the indicated time points, cells were stained for F. tularensis (green) and LAMP-1 (red) and assessed for percent cells infected and the number of bacteria per cell. FIG. 10A provides a series of micrographs showing that CLDC+MPF controlled intracellular replication of F. tularensis. Arrows indicate intracellular F. tularensis. FIG. 10B is a graph showing that uptake of bacteria was assessed four hours after infection. CLDC+MPF treatment did not significantly change the number of bacteria phagocytosed by the macrophages. FIG. 10C is a graph showing that cells treated with CLDC+MPF had fewer cells infected 4 and 24 hours after infection compared to all other treatments and untreated cells. The error bars represent SEM. Data is representative of 4 experiments of similar design.

FIGS. 11A-11C show that CLDC+MPF controls replication of F. tularensis in human macrophages. Human peripheral blood monocytes were differentiated into macrophages and were then treated with 5% Dextrose water (untreated), LVS membrane protein fraction (MPF), cationic liposome DNA complexes (CLDC), or CLDC+MPF overnight. Cells were then infected with F. tularensis Schu4. At the indicated time points cells were stained for F. tularensis (green) and LAMP-1 (red) and assessed for percent cells infected and the number of bacteria per cell. FIG. 11A provides a series of micrographs showing that CLDC+MPF controlled intracellular replication of F. tularensis. Arrows indicate intracellular F. tularensis. FIG. 11B is a graph showing that uptake of bacteria was assessed 4 hours after infection. CLDC+MPF treatment did not significantly change the number of bacteria phagocytosed by the macrophages. FIG. 11C is a graph showing that cells treated with CLDC+MPF had fewer cells infected 24 hours after infection compared to all other treatments and untreated cells. Error bars represent SEM. Data is representative of 4 experiments of similar design.

FIG. 12 is a graph showing that CLDC+MPF inhibits induction of superoxide in mouse macrophages. Mouse bone marrow derived macrophages were treated with 5% Dextrose water (untreated), LVS membrane protein fraction (MPF), cationic liposome DNA complexes (CLDC), or CLDC+MPF overnight. Cells were then infected with F. tularensis Schu4. Uninfected cells treated with PMA served as positive controls for superoxide production. F. tularensis Schu4 induced production of superoxide in mouse macrophages. However, CLDC+MPF treated cells produced significantly less superoxide compared to treated and untreated cells in response to either PMA or infection with F. tularensis Schu4. *=p<0.04. Error bars represent SEM. Data is representative of 4 experiments of similar design.

FIGS. 13A-13D are graphs showing that CLDC+MPF protects against pneumonic tularemia. CLDC+MPF was administrated intravenously 3 days prior to intranasal challenge of mice with 10 CFU F. tularensis Schu4. Mice treated with 5% dextrose water (D5W) served as negative controls. FIG. 13A shows that approximately 80% of mice treated with CLDC+MPF survived lethal Schu4 infection. Four days after infection additional groups of mice were euthanized and assessed for bacterial loads in the spleen and lung, cellular changes in target organs and presence of immunoglobulin directed against F. tularensis Schu4 whole cell lysate (WCL) or purified LPS. FIG. 13B shows that mice treated with CLDC+MPF had significantly fewer bacteria in the spleen and lung compared to mice which received D5W. FIG. 13C show that four days after infection CLDC+MPF treated mice also had significantly more B cells (B220+/IgM+) and plasma cells (B220−/IgM+) cells in the mediastinal lymph node draining the lung compared to mice treated with D5W. FIG. 13C show that there is a correlation with higher numbers of B and plasma cells, treated mice also had higher titers of IgG and IgM directed against F. tularensis Schu4 WCL and LPS compared to mice receiving D5W. Error bars represent SEM. Data is representative of two experiments of similar design.

FIGS. 14A-14C show that CLDC+MPF controls replication of B. pseudomallei in human macrophages. Human peripheral blood monocytes were differentiated into macrophages and were then treated with 5% Dextrose water (untreated) or CLDC+MPF overnight. Cells were then infected with B. pseudomallei. At the indicated time points cells were stained for B. pseudomallei (green) and LAMP-1 (red) and assessed for percent cells infected and the number of bacteria per cell. FIG. 14A is a series of micrographs showing that CLDC+MPF controlled intracellular replication of B. pseudomallei. Arrows indicate intracellular B. pseudomallei. Uptake of bacteria was assessed 1 (FIG. 14B) and 3 (FIGS. 14A and 14B) hours after infection. CLDC+MPF treatment did not significantly change the percent of cells infected. FIG. 14C is a graph showing that cells treated with CLDC+MPF had fewer bacteria per cell 6 hours after infection compared to untreated cells. *=p<0.05. Error bars represent SEM. Data is representative of 2 experiments of similar design.

FIGS. 15A and 15B are graphs showing that CLDC+MPF controls replication of Y. pestis in human macrophages. Human peripheral blood monocytes were differentiated into macrophages and were then treated with 5% Dextrose water (untreated) or CLDC+MPF overnight. Cells were then infected with GFP-expressing Y. pestis (green). At the indicated time points cells were assessed for percent cells infected and the number of bacteria per cell by microscopy. Uptake of bacteria was assessed 2 and 6 hours after infection. FIG. 15A is a graph showing that CLDC+MPF treatment did not significantly change the percent of cells infected. FIG. 15B is a graph showing that cells treated with CLDC+MPF had fewer bacteria per cell 6 hours after infection compared to untreated cells. *=p<0.05. Error bars represent SEM. Data is representative of 2 experiments of similar design.

### DETAILED DESCRIPTION OF THE INVENTION

The invention features compositions and methods that are useful for the treatment or prevention of a pathogen infection. The invention is based, at least in part, on the discovery that infection with a virulent strain of Francisella tularensis could be prevented by administering an immunogenic composition comprising a combination of non-coding DNA and an antigen preparation from F. tularensis in a liposomal formulation. Current treatments for pneumonic tularemia require administration of antibiotics within the first few days of infection. If antibiotic therapy is not undertaken at this time, the vast majority of patients die from the infection. The utility of the existing therapeutic regimen is severely limited by the fact that most subjects are unaware that they have contracted a Francisella tularensis infection during the period when antibiotic therapy is effective.

In view of the rapid lethality and infectiousness of even a low dose (e.g., 10 CFU) of F. tularensis, both the U.S. and the former Soviet Union weaponized the pathogen. In the event of a bioterror attack, first line antibiotics are unlikely to be effective against genetically engineered antibiotic resistant strains of F. tularensis. Furthermore, the development of immunotherapeutics against Francisella tularensis has been hampered by the fact that virulent strains of Francisella suppress the ability of the host to respond to pro-inflammatory stimuli.

In response to this need for a rapid, non-antibiotic based, prophylaxis to combat pneumonic tularemia, an immunogenic composition was developed which comprises non-coding DNA and antigen preparations from F. tularensis combined in liposomes (CMF). CMF delivered three days prior to aerosol challenge with F. tularensis Schu4 (a virulent Type A strain) effectively protected approximately 80% of infected animals. The nature of this protection appeared to be both antibody and specific activation of infected effector cells. This is the first demonstration of rapid protection against aerosolized Type A F. tularensis. Although the examples particularly describe the use of cationic liposome complexes comprising non-coding DNA and membrane fraction from F. tularensis, the invention is not so limited. Based on the success of this prophylactic approach against a particularly virulent pathogen, one of skill in the art would expect it to be at least as effective for the prevention or treatment of other pathogens. The invention provides a novel, effective tularemia prophylaxis and therapy, and the methods of the invention can be applied for the treatment of a variety of other infectious diseases. In general, the method involves providing a non-coding DNA (e.g., a DNA comprising a high percentage of guanosines or cytosine residues) and a pathogen antigen (e.g., a membrane fraction or other antigen containing fraction derived from a pathogen) together with a cationic liposome. In one embodiment, the cationic liposome comprises 1-[2-(9(2)-octadecenoyloxy)ethyl]-2-(8(2)-heptadecenyl)-3-(2-hydroxyethyl)-midizolinium chloride.

In particular embodiments, the invention provides for the treatment of bacterial infections, including infections with gram negative and gram positive bacteria, which serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Burkholderia sps, Borellia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia ssp, Yersinia pestis and Actinomyces israelli. Accordingly, the invention provides for a cationic liposome DNA complex that contains a gram negative or gram positive bacterial antigen.

In other embodiments, the antigen or membrane fraction is derived from an infectious organism (i.e., protist). Such organisms include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

In still other embodiments, the pathogen antigen is derived from a pathogenic fungus, including, without limitation, Alternaria, Aspergillus, Basidiobolus, Bipolaris, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, and Candida lusitaniae, Coccidioides, Cladophialophora, Cryptococcus, Cunninghamella, Curvularia, Exophiala, Fonsecaea, Histoplasma, Madurella, Malassezia, Plastomyces, Rhodotorula, Scedosporium, Scopulariopsis, Sporobolomyces, Tinea, and Trichosporon.

Fungi, including, but not limited to Candida, cause invasive diseases in hosts with altered immunity, such as patients with HIV infection, organ or bone marrow transplants, or neutropenia following cancer immunotherapy. There are approximately 200 species of the genus Candida, but nine cause the great majority of human infections. They are C. albicans, C. krusei, C. glabrata (formerly called Torulopsis glabrata), C. parapsilosis, C. tropicalis, C. pseudotropicalis, C. guilliermondii, C. dubliniensis, and C. lusitaniae. They cause infections of the mucous membranes, for example, thrush, esophagitis, and vagititis; skin, for example, intertrigo, balanitis, and generalized candidiasis; blood stream infections, for example, candidemia; and deep organ infections, for example, hepatosplenic candidiasis, urinary tract candidiasis, arthritis, endocarditis, and endophthamitis.

In still other embodiments, the methods of the invention can be used to treat or prevent a viral infection. Accordingly, the invention provides a composition comprising antigen or membrane fraction is derived from a virus. Exemplary viral pathogens include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

### Vaccine Production

The invention also provides for a method of inducing an immunological response in a subject, particularly a human, which comprises inoculating the individual with a composition of the invention (e.g., CLCD in combination with a pathogen derived antigen, including a membrane fraction), in a suitable carrier for the purpose of inducing an immune response to protect said subject from infection with a pathogen. The administration of this immunological composition may be used either therapeutically in individuals already experiencing an pathogen infection, or may be used prophylactically to prevent a pathogen infection.

Therapeutic vaccines may reduce or alleviate a symptom associated with a pathogen infection, such as the severity of tularemia. In some cases, a therapeutic vaccine will enhance the immune response of an individual infected with the pathogen. Prophylactic vaccines may be used to prevent or reduce the probability that a subject (e.g., a human) will be infected with the pathogen. In some embodiments, a vaccine prevents the transmission of the pathogen from an infected subject to an uninfected subject.

The preparation of membrane fractions that contain pathogen derived antigens is known to one skilled in the art, and is described herein. In other embodiments, the pathogen derived antigen is a polypeptide that may be used as an antigen for vaccination in combination with the CLDC. Methods for making CLDC are described, for example, in U.S. Pat. No. 6,696,086, which is hereby incorporated by reference in its entirety.

For example, Francisella membrane fractions, polypeptides, or fragments or variants thereof are delivered in combination with CLDC in vivo in order to induce an immune response. The polypeptides might be fused to a recombinant protein that stabilizes the polypeptide of the invention, aids in its solubilization, facilitates its production or purification, or acts as an adjuvant by providing additional stimulation of the immune system.

Typically vaccines are prepared in an injectable form, either as a liquid solution or as a suspension. Solid forms suitable for injection may also be prepared as emulsions, or with the membrane fraction or polypeptides encapsulated in liposomes. Vaccine antigens are usually combined with a pharmaceutically acceptable carrier, which includes any carrier that does not induce the production of antibodies harmful to the individual receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

Adjuvants are immunostimulating agents that enhance vaccine effectiveness. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Immunogenic compositions, i.e. the antigen, pharmaceutically acceptable carrier and adjuvant, also typically contain diluents, such as water, saline, glycerol, ethanol. Auxiliary substances may also be present, such as wetting or emulsifying agents, pH buffering substances, and the like. Antigens (e.g., pathogen derived polypeptides) may be formulated into the vaccine as neutral or salt forms. The vaccines are typically administered parenterally, by injection; such injection may be either subcutaneously or intramuscularly. Additional formulations are suitable for other forms of administration, such as by suppository or orally. Oral compositions may be administered as a solution, suspension, tablet, pill, capsule, or sustained release formulation.

In addition, the vaccine can also be administered to individuals to generate polyclonal antibodies (purified or isolated from serum using standard methods) that may be used to passively immunize an individual. These polyclonal antibodies can also serve as immunochemical reagents.

Vaccines are administered in a manner compatible with the dose formulation. The immunogenic composition of the vaccine comprises an immunologically effective amount of the antigenic polypeptides and other previously mentioned components. By an immunologically effective amount is meant a single dose, or a vaccine administered in a multiple dose schedule, that is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgment of the practitioner.

### Vaccine Formulations

A therapeutic composition of the present invention includes a liposome delivery vehicle. According to the present invention, a liposome delivery vehicle comprises a lipid composition that is capable of preferentially delivering a therapeutic composition of the present invention to tissues in a mammal to induce an immune response. Effective immune activation at immunologically active organs is provided by the composition without the aid of additional targeting mechanisms. A preferred liposome delivery vehicle of the present invention is between about 100 and 500 nanometers (nm), more preferably between about 150 and 450 nm and even more preferably between about 200 and 400 nm in diameter. As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

Suitable liposomes for use with the present invention include any liposomes commonly used in, for example, gene delivery methods known to those of skill in the art. Preferred liposome delivery vehicles comprise multilamellar vesicle (MLV) lipids and extruded lipids. Methods for preparation of MLV's are well known in the art and are described, for example, in the U.S. Pat. No. 6,696,083. According to the present invention, “extruded lipids” are lipids which are prepared similarly to MLV lipids, but which are subsequently extruded through filters of decreasing size, as described in Templeton et al., 1997, Nature Biotech., 15:647-652, which is incorporated herein by reference in its entirety. Although small unilamellar vesicle (SUV) lipids can be used in the composition and method of the present invention, multilamellar vesicle lipids are significantly more immunostimulatory. More preferred liposome delivery vehicles comprise liposomes having a polycationic lipid composition (i.e., cationic liposomes) and/or liposomes having a cholesterol backbone conjugated to polyethylene glycol. Preferred cationic liposome compositions include, but are not limited to DOTMA and cholesterol, DOTAP and cholesterol, DOTIM and cholesterol, and DDAB and cholesterol.

Complexing a liposome with a nucleic acid molecule and a pathogen derived antigen of the present invention can be achieved using methods standard in the art. According to the present invention a cationic lipid:DNA complex is also referred to herein as a CLDC. A suitable concentration of a nucleic acid molecule of the present invention to add to a liposome includes a concentration effective for delivering a sufficient amount of nucleic acid molecule into a mammal such that a systemic immune response is elicited. Preferably, from about 0.1 μg to about 10 μg of nucleic acid molecule of the present invention is combined with about 8 nmol liposomes, more preferably from about 0.5 μg to about 5 μg of nucleic acid molecule is combined with about 8 nmol liposomes, and even more preferably about 1.0 μg of nucleic acid molecule is combined with about 8 nmol liposomes. In one embodiment, the ratio of nucleic acids to lipids (μg nucleic acid:nmol lipids) in a composition of the present invention is preferably at least about 1:1 nucleic acid:lipid by weight (i.e., 1 μg nucleic acid:1 nmol lipid), and more preferably, at least about 1:5, and more preferably at least about 1:10, and even more preferably at least about 1:20. Ratios expressed herein are based on the amount of cationic lipid in the composition, and not on the total amount of lipid in the composition. In another embodiment, the ratio of nucleic acids to lipids in a composition of the present invention is preferably from about 1:1 to about 1:64 nucleic acid:lipid by weight; and more preferably, from about 1:5 to about 1:50 nucleic acid:lipid by weight; and even more preferably, from about 1:10 to about 1:40 nucleic acid:lipid by weight; and even more preferably, from about 1:15 to about 1:30 nucleic acid:lipid by weight. Another particularly preferred ratio of nucleic acid:lipid is from about 1:8 to 1:16, with 1:8 to 1:32 being more preferred. Typically, while non-systemic routes of nucleic acid administration (i.e., intramuscular, intratracheal, intradermal) would use a ratio of about 1:1 to about 1:3, systemic routes of administration according to the present invention can use much less nucleic acid as compared to lipid and achieve equivalent or better results than non-systemic routes.

Complexing a pathogen derived antigen (e.g., membrane fraction) with a liposome and nucleic acid molecule is accomplished in a straightforward manner. The antigen can be complexed with a preformed complex of nucleic acid and liposome, or it can be complexed with the liposome at the same time as the nucleic acid molecule. The pathogen derived antigen (e.g., membrane fraction) can be effectively complexed with the liposome simply by gently mixing the pathogen derived antigen (e.g., membrane fraction) and the liposome (and the nucleic acid) together, preferably in a suitable excipient (e.g., 5-10% sucrose or 5-10% lactose). The pathogen derived antigen (e.g., membrane fraction) can also be incorporated into the liposome as the liposome is formulated (e.g., rehydrated). The pathogen derived antigen (e.g., membrane fraction) can be mixed with the preformed lipid and nucleic acid complexes; mixed with the preformed lipid, followed by adding the nucleic acid; or can be mixed with the nucleic acid and then, together, added to preformed liposomes.

A suitable concentration of an pathogen derived antigen (e.g., membrane fraction) to add to a liposome includes a concentration effective for delivering a sufficient amount of pathogen derived antigen (e.g., membrane fraction) into a mammal such that an pathogen derived antigen (e.g., membrane fraction)-specific immune response is elicited, at least at or near the site of administration, and preferably, systemically. Preferably, from about 1 μg antigen per individual mammal to about 1 mg immunogen per individual mammal is combined with about 8 nmol liposomes (or other suitable amount of liposomes which can be determined by the skilled artisan), more preferably from about 1 μg immunogen per individual mammal to about 100 μg immunogen per individual mammal is combined with about 8 nmol liposomes, and even more preferably from about 1 μg immunogen per individual mammal to about 10 μg immunogen per individual mammal is combined with about 8 nmol liposomes. In one embodiment, at least about 0.1 μg immunogen per individual mammal, and more preferably at least about 1 μg antigen per individual mammal, and more preferably, at least about 5 μg antigen per individual mammal, and more preferably at least about 10 μg antigen per individual mammal is added to a liposome composition of the present invention.

In another embodiment of the present invention, a therapeutic composition further comprises a pharmaceutically acceptable excipient. Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Particularly preferred excipients include non-ionic diluents, with a preferred non-ionic buffer being 5% dextrose in water.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Therapeutic compositions of the present invention can be sterilized by conventional methods and/or lyophilized.

According to the present invention, an effective administration protocol (i.e., administering a therapeutic composition in an effective manner) comprises suitable dose parameters and modes of administration that result in elicitation of an immune response in a mammal that has a disease, preferably so that the mammal is protected from the disease. Effective dose parameters can be determined using methods standard in the art for a particular disease. Such methods include, for example, determination of survival rates, side effects (i.e., toxicity) and progression or regression of disease. In particular, the effectiveness of dose parameters of a therapeutic composition of the present invention when treating an infectious disease can be determined by assessing an immune response or by assessing a reduction in the growth, replication, survival of a pathogen.

In accordance with the present invention, a suitable single dose size is a dose that is capable of eliciting an immune response in a mammal with a disease when administered one or more times over a suitable time period. Doses can vary depending upon the disease being treated. Doses of a therapeutic composition of the present invention suitable for use with intravenous or intraperitoneal administration techniques can be used by one of skill in the art to determine appropriate single dose sizes for systemic administration based on the size of a mammal.

In a preferred embodiment, an appropriate single dose of a nucleic acid:liposome complex of the present invention is from about 0.1 μg to about 100 μg per kg body weight of the mammal to which the complex is being administered. In another embodiment, an appropriate single dose is from about 1 μg to about 10 μg per kg body weight. In another embodiment, an appropriate single dose of nucleic acid:lipid complex is at least about 0.1 μg of nucleic acid to the mammal, more preferably at least about 1 μg of nucleic acid, even more preferably at least about 10 μg of nucleic acid, even more preferably at least about 50 μg of nucleic acid, and even more preferably at least about 100 μg of nucleic acid to the mammal.

A suitable single dose of a therapeutic composition of the present invention to elicit a systemic, immune response in a mammal is a sufficient amount of a nucleic acid molecule complexed to a liposome delivery vehicle, when administered intravenously or intraperitoneally, to elicit a cellular and/or humoral immune response in vivo in a mammal, as compared to a mammal which has not been administered with the therapeutic composition of the present invention (i.e., a control mammal). Preferred dosages of nucleic acid molecules to be included in a nucleic acid:lipid complex of the present invention have been discussed, above.

According to the present invention, a single dose of a therapeutic composition useful to elicit an immune response against an infectious disease may be administered. It will be obvious to one of skill in the art that the number of doses administered to a mammal is dependent upon the extent of the disease and the response of an individual patient to the treatment. Thus, it is within the scope of the present invention that a suitable number of doses includes any number required to treat a given infectious disease.

Elicitation of an immune response using the compositions and methods of the present invention typically includes an initial administration of the therapeutic composition, followed by booster immunizations at 3-4 weeks after the initial administration, optionally followed by subsequent booster immunizations every 3-4 weeks after the first booster, as needed to treat a disease according to the present invention. A preferred number of doses of a therapeutic composition comprising nucleic acid molecule complexed with a liposome delivery vehicle in order to elicit an immune response against a pathogen, is from about 2 to about 10 administrations patient, more preferably from about 3 to about 8 administrations per patient, and even more preferably from about 3 to about 7 administrations per patient. Preferably, such administrations are given once every 3-4 weeks, as described above, until signs of remission appear, and then once a month until the disease is gone.

According to the method of the present invention, a therapeutic or prophylactic composition is administered by intravenous or intraperitoneal injection, and preferably, intravenously. Intravenous injections can be performed using methods standard in the art. According to the method of the present invention, administration of the nucleic acid:lipid complexes can be at any site in the mammal wherein systemic administration (i.e., intravenous or intraperitoneal administration) is possible, particularly when the liposome delivery vehicle comprises cationic liposomes. Administration at any site in a mammal will elicit a potent immune response when either intravenous or intraperitoneal administration is used, and particularly, when intravenous administration is used. Suitable sites for administration include sites in which the target site for immune activation is not restricted to the first organ having a capillary bed proximal to the site of administration (i.e., compositions can be administered at an administration site that is distal to the target immunization site). In other words, for example, intravenous administration of a composition of the present invention which is used to treat a pathogen infection in a mammal can be administered intravenously at any site in the mammal and will still elicit a strong immune response and be efficacious at reducing or eliminating the infection. For immunization and immune activation, intraperitoneal administration is also a suitable mode of administration.

### Screening Assays

The invention provides methods for enhancing an immune response by administering a composition comprising a cationic liposome complex with DNA (CLDC) together with a viral antigen (e.g., a membrane fraction comprising a viral antigen). While the Examples described herein specifically discuss the use of a membrane fraction derived from Franciscella, one skilled in the art understands that the methods of the invention are not so limited. Virtually any antigenic agent (e.g., polypeptide) derived from a pathogen (e.g., gram negative bacterium, gram positive bacterium, fungus, virus, protist, or parasite) may be employed in the methods of the invention.

Methods of the invention are useful for the high-throughput low-cost screening of candidate agents that increase an immune response for the treatment or prevention of an infectious disease. A candidate agent (e.g., pathogen derived antigen, including a crude membrane fraction) that induces an immune response in a subject or in an in vitro assay is identified as useful in the methods of the invention. In one embodiment, the candidate agent is identified as inducing the production of proinflammatory cytokines from antigen presenting cells, for increasing the survival of a subject in a pathogen challenge assay, to reduce pathogen growth, survival, or replication, to induce the proliferation of a plasma cell or the proliferation of another immune cell (B cell, memory T cell, activation of macrophages and dendritic cells, degranulation, or activation of granulocytes). One skilled in the art appreciates that the effects of a candidate agent on a cell is typically compared to a corresponding control cell not contacted with the candidate agent. Thus, the screening methods include comparing the proliferation of a plasma cell (or progenitor cell) in an animal contacted by a candidate agent to the proliferation of such cells in an untreated control animal.

In one working example, one or more candidate agents are added at varying concentrations to the culture medium containing an immune cell (e.g., plasma cell of antigen presenting cell). An agent that promotes an immune response in the cell or promotes proliferation of the cell is considered useful in the invention. An immune response may be assayed by identifying an increase in cytokine production, measuring an increase in activated B cells, measuring an increase in antibody production (e.g., IgG or IgM). An agent identified according to the methods of the invention may be used, for example, as a therapeutic to prevent, delay, ameliorate, stabilize, or treat a pathogen infection. Once identified, agents of the invention may be used to increase an adaptive or innate immune response in a patient in need thereof. In particular, an agent identified according to a method of the invention is locally or systemically delivered to increase an immune response in a subject for the prevention or treatment of a pathogen infection. Such agents may be used, for example, as a therapeutic to combat the pathogenicity of an infectious pathogen. Optionally, agents identified in any of the above-described assays may be confirmed as useful in conferring protection against the development of a pathogen infection in any standard animal model (e.g., a F. tularensis challenge, LPS, challenges with Listeria monocytogenes, Yersinia pestis, Nisseria meningitidis, influenzae, Venezualan equine encephalitis, or RMA-S lymphoma challenge) and, if successful, identified agents may be used as anti-pathogen therapeutics.

Accordingly, the present invention provides methods of treating pathogen related diseases and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a pathogen-related disease or disorder or symptom thereof. The method includes the step of administering to the mammal a therapeutic amount of an amount of a compound (e.g., CLDC in combination with a pathogen antigen) herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which a pathogen infection may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a pathogen infection, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Combination Therapies

Compositions of the invention useful for the treatment of a pathogen infection may, if desired, be administered in combination with any standard therapy known in the art. For example, the immunogenic compositions of the invention may, if desired, be administered in combination with an agent that reduces the survival of a pathogen, including but not limited to Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride, Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz: Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium: Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride.

### Kits

The invention provides kits for the treatment or prevention of a pathogen infection. In one embodiment, the kit includes a therapeutic or prophylactic composition containing an effective amount of a liposome formulation, a non-coding DNA, and a pathogen derived antigen in unit dosage form. In some embodiments, the kit comprises a sterile container, which contains a therapeutic or prophylactic cellular composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired a cell of the invention is provided together with instructions for administering the agent to a subject having or at risk of developing a pathogen infection or infectious disease, such as tularemia. The instructions will generally include information about the use of the composition for the treatment or prevention of a pathogen infection. In other embodiments, the instructions include at least one of the following: description of the therapeutic agent; dosage schedule and administration for treatment or prevention of a pathogen infection or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Vaccination with CLDC and LVS Membrane Antigens Protected Infected Animals

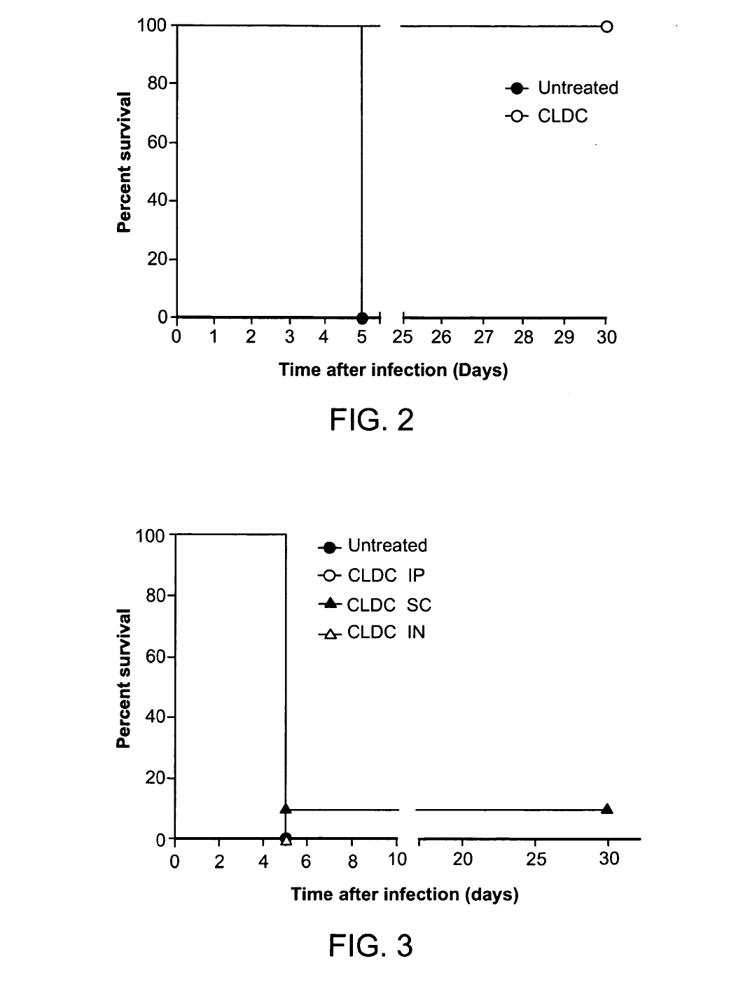

Current treatment of pneumonic tularemia requires administration of antibiotics within the first few days of infection. This regime poses two serious limitations: (i) many individuals are unaware they have contracted the infection during this time; (ii) in the event of a bioterror attack many first line antibiotics may not be effective against genetically engineered strains of F. tularensis that are resistant to these drugs. Furthermore, development of immunotherapeutics has been hampered by the fact that virulent strains of Francisella suppress the ability of the host to respond to pro-inflammatory stimuli. Interestingly, a complex of cationic liposome and non-coding DNA (CLDC) protected against attenuated (LVS) (FIG. 2), but did not provide protection against fully virulent (Schu4) strains of F. tularensis (FIG. 3).

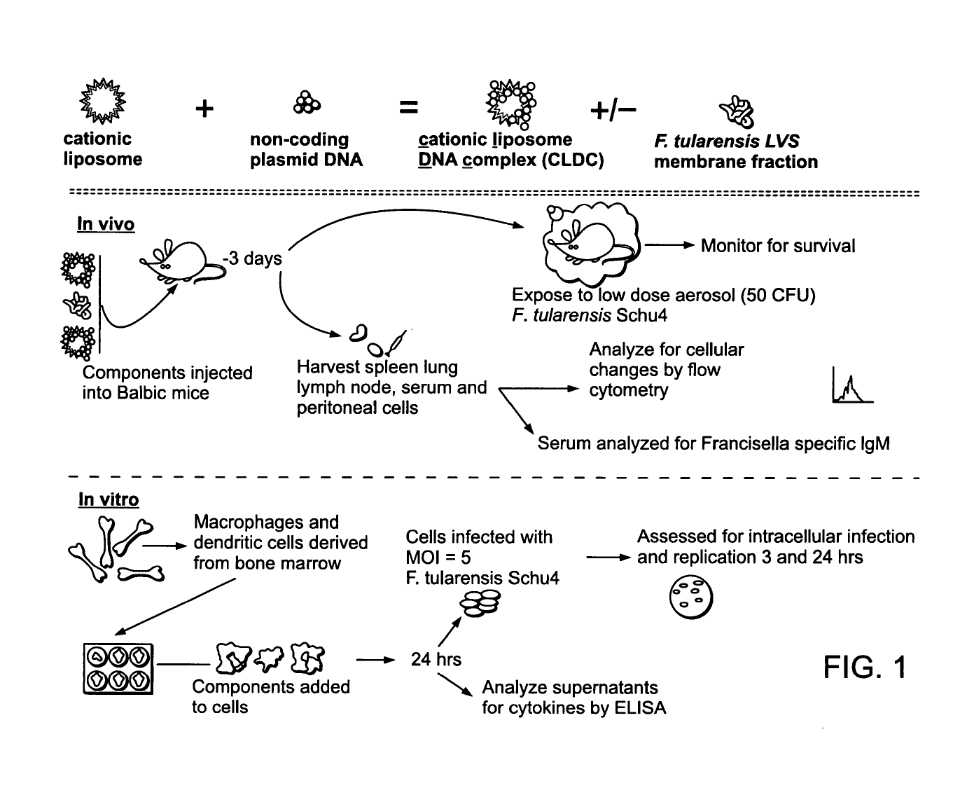

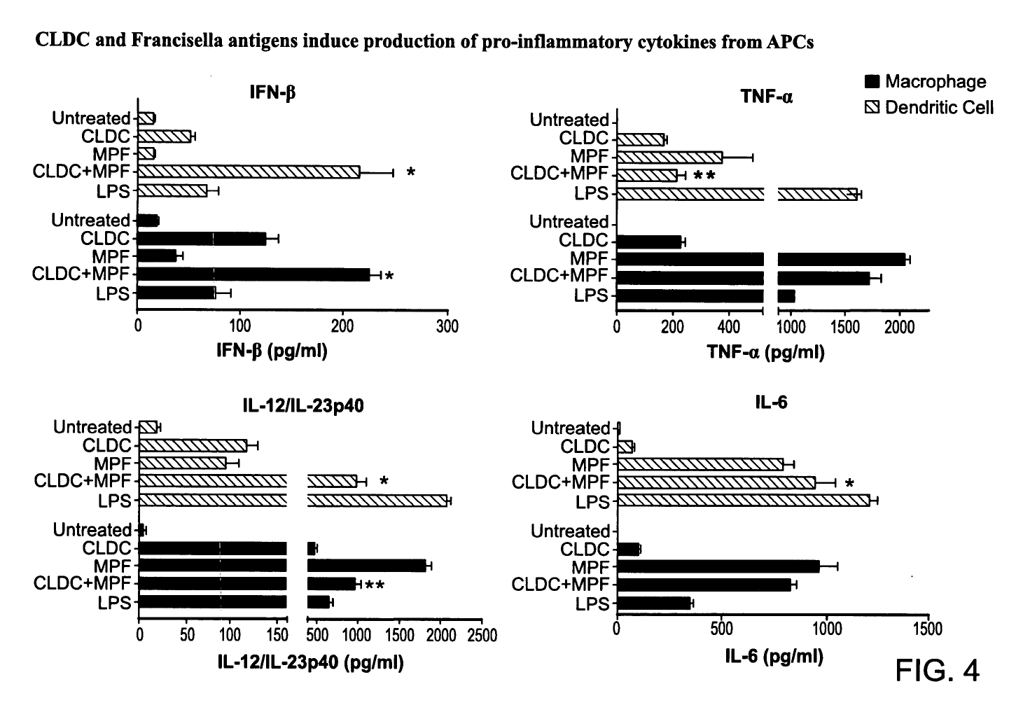

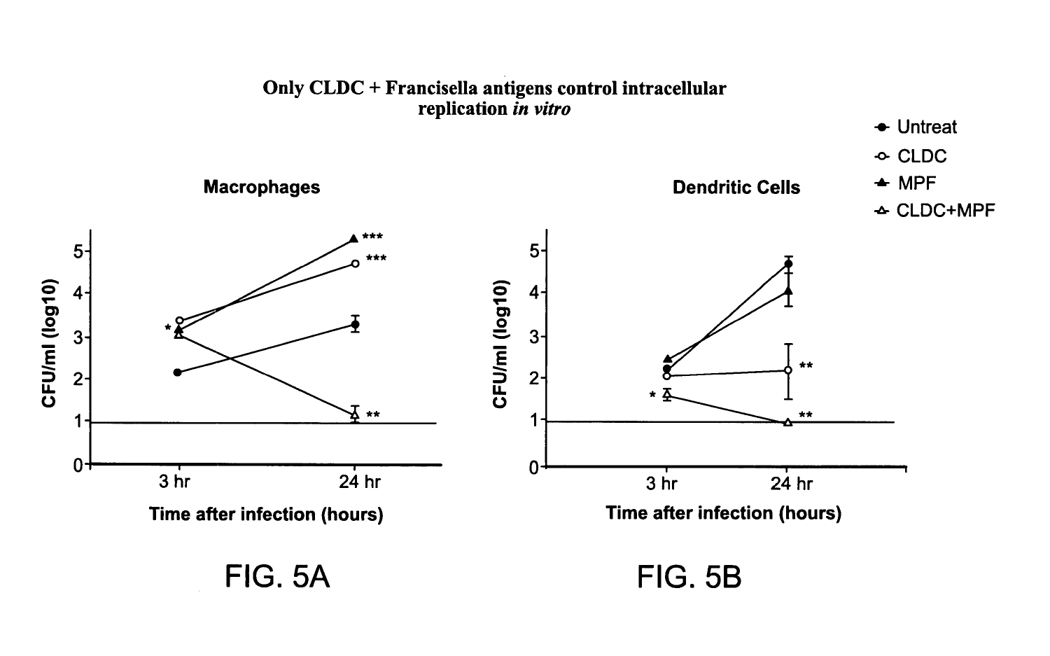

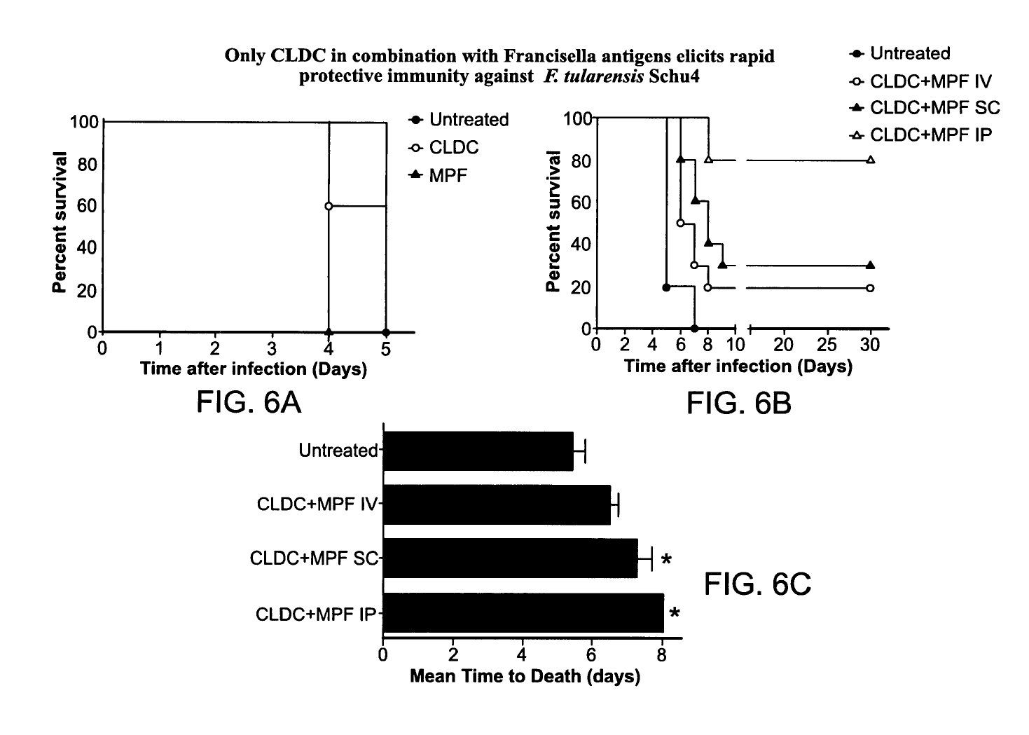

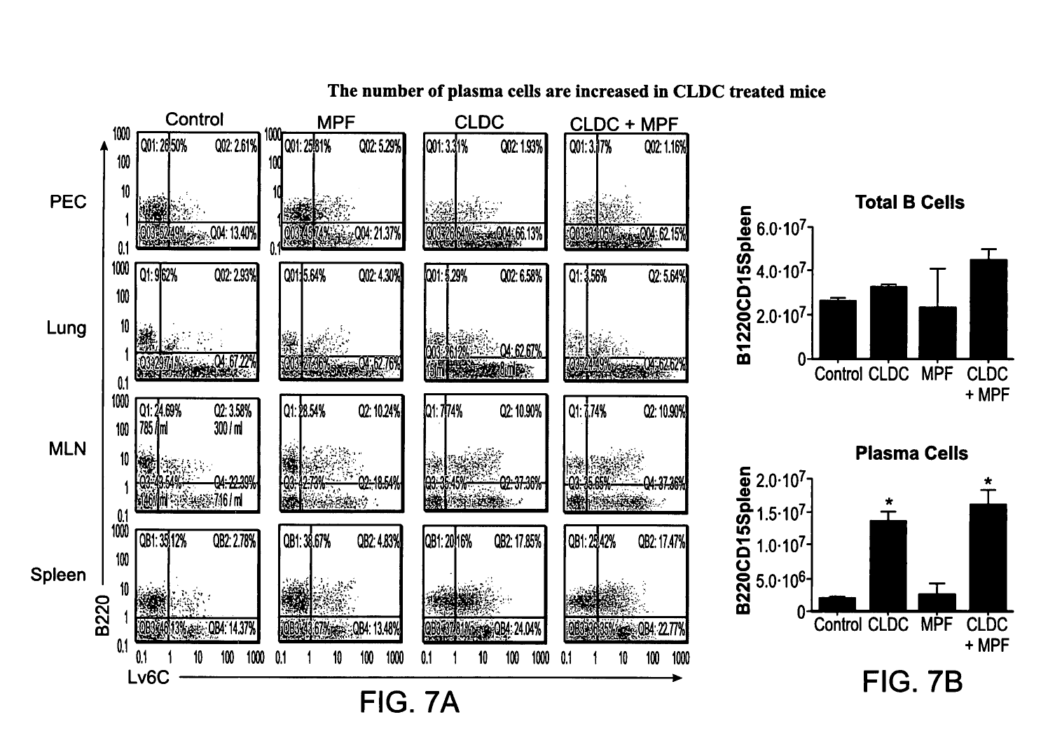

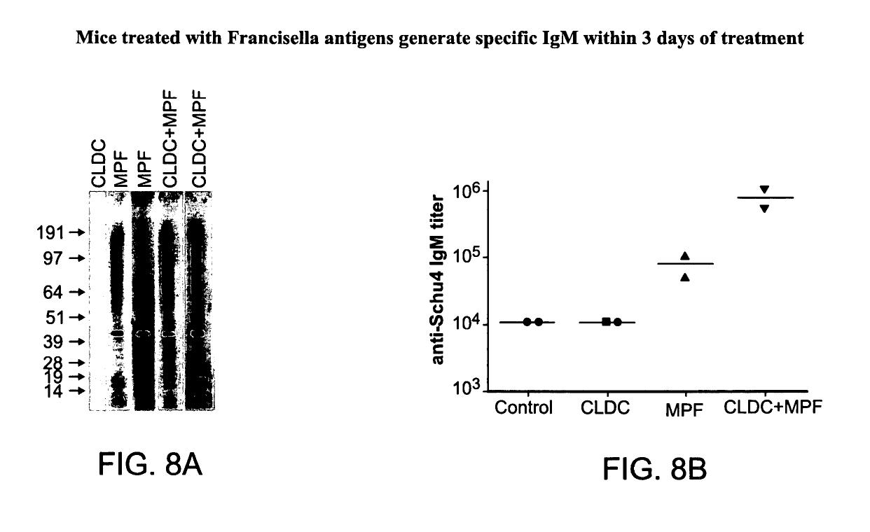

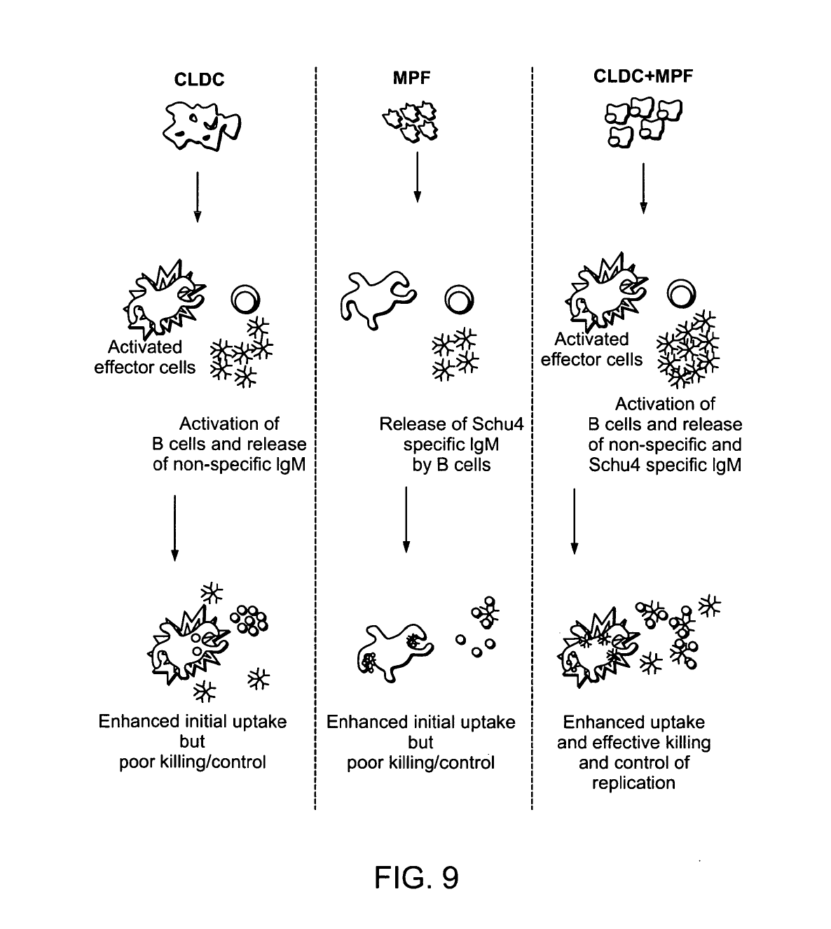

In response to the need for a rapid, non-antibiotic based, prophylaxis to combat pneumonic tularemia, a three component prophylaxis consisting of non-coding DNA and crude antigen preparations from F. tularensis combined in liposomes (CMF) was developed (FIG. 1). CLDC in combination with membrane antigens from LVS enabled target cells to control Schu4 in vitro (FIGS. 4 and 5). This composition was delivered to mice three days prior to aerosol challenge with F. tularensis Schu4 (a virulent Type A strain). This prophylaxis effectively protected approximately 80% of infected animals. Specifically, CLDC+LVS membrane antigens administered only 3 days prior to challenge increased mean time to death and survival following low dose aerosol challenge with Schu4 (FIG. 6). In addition, CLDC increased plasma cells within 3 days of administration in vivo (FIG. 7). Furthermore, LVS membrane antigens elicited production of Schu4 specific IgM within 3 days of administration (FIGS. 8A and 8B). Thus, the nature of the prophylactic protection appears to be both antibody and specific activation of infected effector cells. This is the first demonstration of rapid protection against aerosolized Type A F. tularensis. Accordingly, the invention provides a novel, effective tularemia prophylaxis and therapy (FIG. 9).

### Example 2

### CLDC+MPF Compositions Provide Prophylaxis Against Bacterial and Viral Diseases

The CLDC+MPF compound is useful as a prophylaxis, particularly a short term prophylaxis, against bacterial and viral diseases. For example, the cells that infiltrate specific target tissues were characterized following administration of treatment before and after infection with virulent F. tularensis Schu4. This has provided for the identification of the specific cellular killing mechanisms in mouse and human cells induced by CLDC+MPF treatment that are associated with control of bacterial replication. These results indicate that this composition would be effective against other microbial pathogens including Burkholderia pseudomallei, Yersinia pestis and Influenza virus.

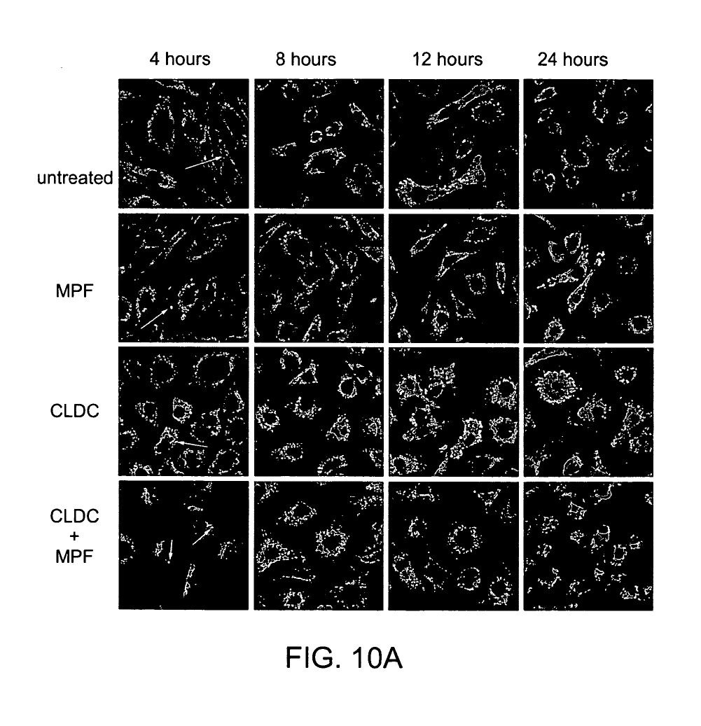

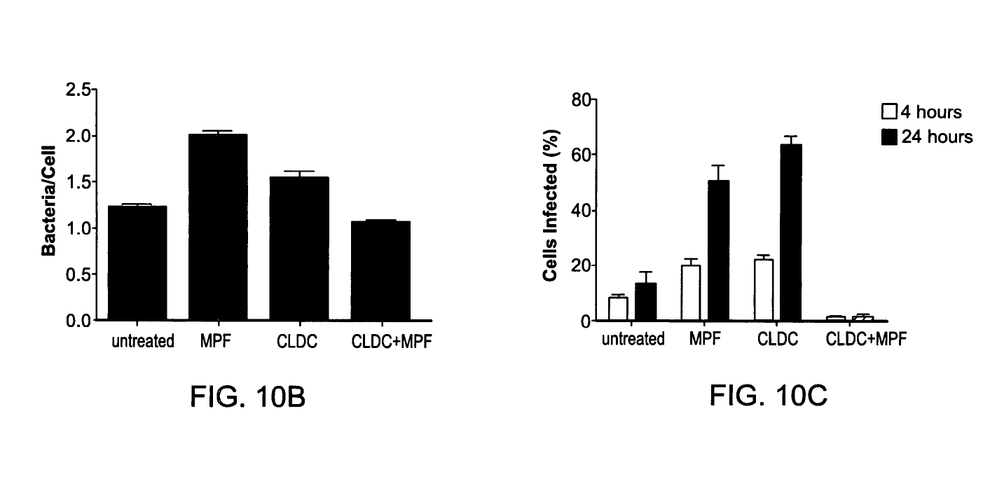

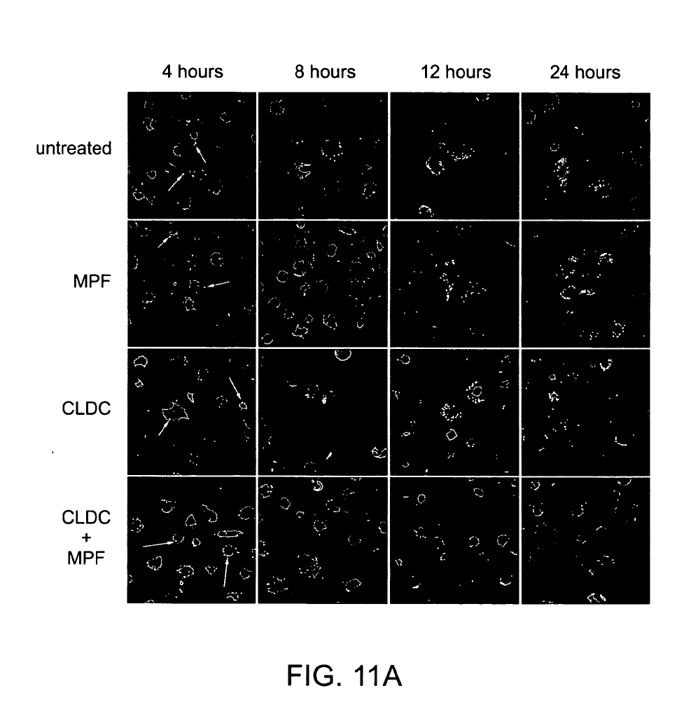

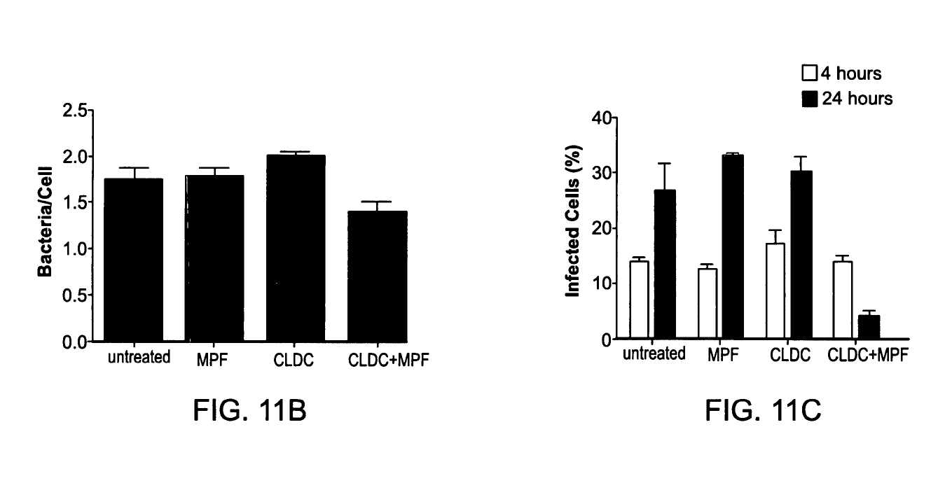

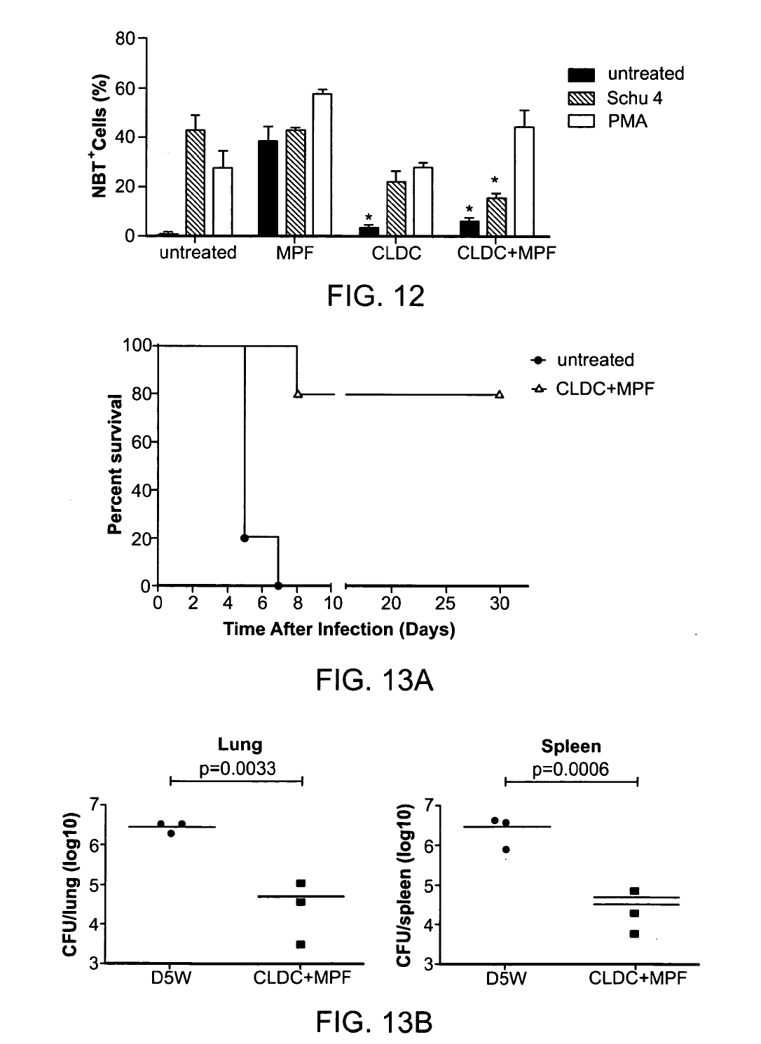

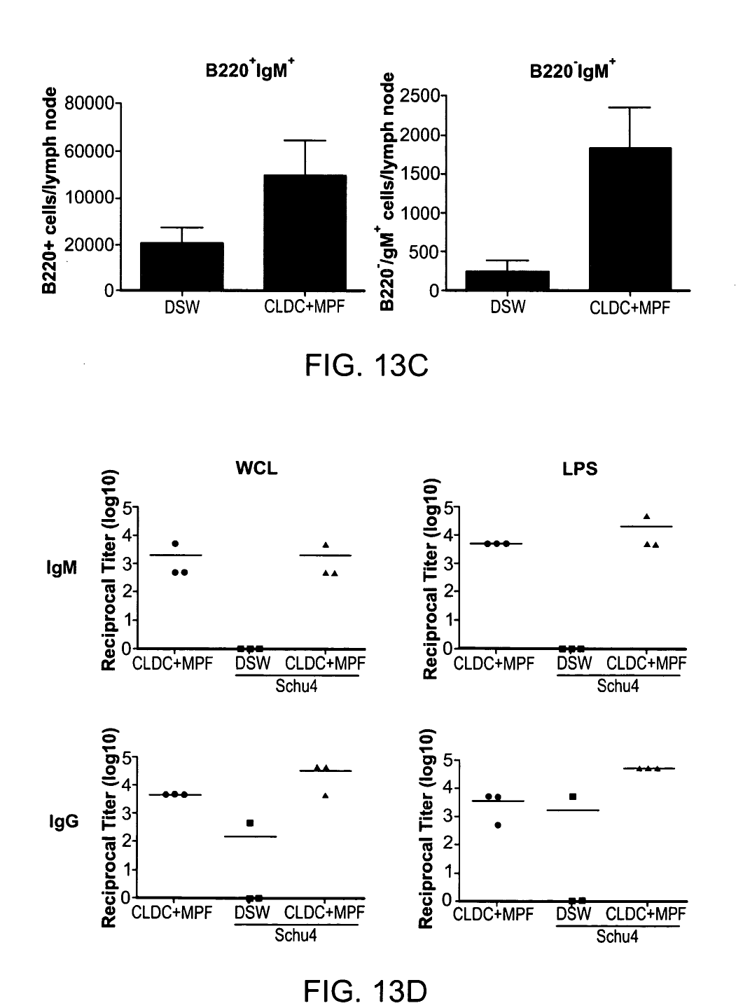

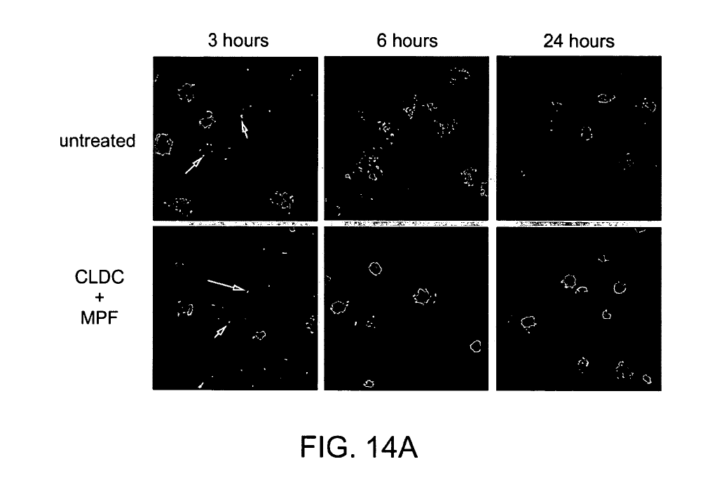

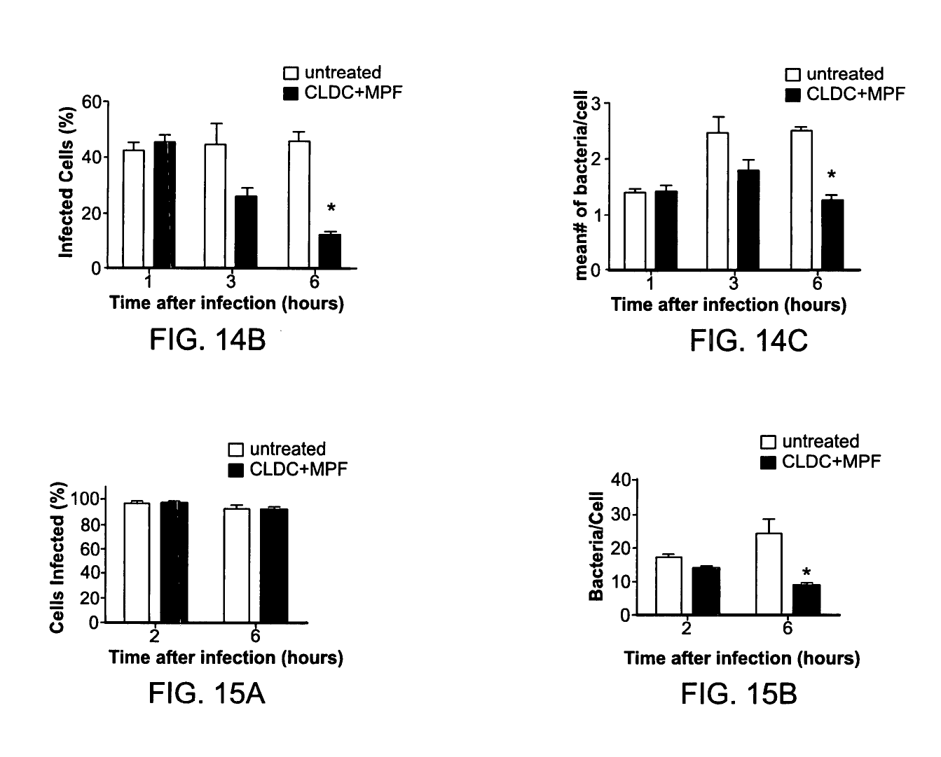

In the development of this compound for use against infection it was shown that vaccination with the compound inhibited Francisella tularensis intracellular growth (FIG. 10 and FIG. 11). This result provided for the identification of at least one oxygen radical pathway involved in this inhibition (FIG. 12). In addition, extensive in vivo experiment indicate that control of virulent F. tularensis is tightly correlated with expansion of plasma cells in the draining lymph node and induction of Francisella specific antibodies is critical for in vivo (FIG. 13). Furthermore, it was also recently demonstrated that CLDC+MPF efficiently controls replication of both Burkholderia pseudomallei (the causative agent of mellidosis) and Yersinia pestis (the causative agent of Plague) in macrophages in vitro (FIGS. 14 and 15). These last series of experiments performed with B. pseudomallei and Y. pestis are especially important because they indicate that CLDC+MPF will be an effective prophylaxis against multiple infectious diseases, including bacterial meningitis, influenza and pneumonicoccal mediated diseases.

The following methods and materials were used to obtain the results described herein.

### Preparation of Cationic Liposomes

Liposomes were prepared by dissolving the cationic lipid octadecenolyoxy[ethyl-2-heptadecenyl-3 hydroxyethyl]imidazolinium chloride (Sigma-Aldrich Chemical) and cholesterol (Avanti Polar Lipids) in chloroform and adding equimolar concentrations to round-bottom, 15-ml glass tubes to a final concentration of 2 mM. The solution was then dried overnight in a vacuum desiccator to a thin film. The lipids were rehydrated in 5% dextrose in water at 50° C. for 50 minutes, followed by incubation for 2 hours at room temperature. The liposomes were then extruded through a series of 1-, 0.45-, and 0.20-μm filters to form the final liposomes, as descried previously (Templeton, N. S., D. D. Lasic, P. M. Frederik, H. H. Strey, D. D. Roberts, G. N. Pavlakis. 1997. Improved DNA: liposome complexes for increased systemic delivery and gene expression. Nat. Biotechnol. 15: 647-652).

To formulate the cationic liposome complexes (CLDC), liposomes and non-coding plasmid DNA were prepared by first diluting the liposomes in 5% dextrose in water at a concentration of 100 μl of liposomes per 1 ml of dextrose solution. Next, low endotoxin content, non-coding plasmid DNA (pMB75.6) (Althea Technologies) were added with gentle pipetting to the liposome solution at a final concentration of 100 μg of agonist per milliliter of liposome solution.

To formulate the CLDC-antigen complexes, 10 ug of LVS membrane protein fraction (MPF) are mixed by gently pipetting MPF with CLDC. CLDC-MPF were prepared at room temperature and administered within 30 minutes of preparation.

### LVS Fractionation Protocol

LVS bacteria were centrifuged to form LVS cell pellets (e.g., Pellet 1: 4.90 g, Pellet 2: 5.84 g, Pellet 3: 5.13 g) and frozen. 50 ml of breaking buffer (PBS pH 7.4, 60 μg DNase, 60 μg RNase, 50 μg of lysozyme, 1 complete EDTA-free protease inhibitor cocktail tablet) was made fresh and kept on ice. LVS cell pellets were thawed on ice and resuspended in 1 ml of breaking buffer for every 1 g of LVS cells. Pellets were sonicated 9 times (60 sec on, 60 sec off). The sample was centrifuged at 3700 rpm for 20 minutes to pellet unbroken cells and the supernatant was removed. This was done twice and supernatants removed were pooled. The supernatant was centrifuged at 100,000×g for 4 hours at 4° C. The supernatant was removed and labeled as soluble fraction. Both the supernatant and remaining pellet (membrane/outer envelope) were frozen at −80° C.

Pellet and supernatant were thawed on ice the next day. The membrane pellet was resuspended in ˜40 ml of fresh breaking buffer and both the cytosol and membrane fractions were centrifuged again as described above. The supernatant was removed from the membrane pellet and the pellet washed with breaking buffer. The pellet was resuspended in 10 ml of breaking buffer. Both cytosol and membrane fractions were dialyzed against 10 mM ammonium bicarbonate buffer for 24 hours at 4° C. using a 3500 MWCO tubing. Protein content was determined using the BCA assay, which was performed on both fractions (see results below). Fractions were run on SDS-page gel and silver stained as well as probed for LPS. Aliquots of 1 mg/ml were made for both fractions and frozen at −80° C.

### BCA Results:

Cytosol: ˜2.64 mg/ml

Membrane: ˜7.21 mg/ml

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A vaccine comprising a liposome, a non-coding nucleic acid molecule, and a bacterial, fungal, or parasitic antigen in a pharmaceutically acceptable excipient, wherein the vaccine induces a protective immune response in a vaccinated mammal.

2. The vaccine of claim 1, wherein the bacterial, fungal, or parasitic antigen is provided in a membrane fraction.

3. The vaccine of claim 1, wherein the liposome is a cationic liposome.

4. The vaccine of claim 1, wherein the vaccine prevents or treats a bacterial, fungal, or parasitic infection of the mammal.

5. The vaccine of claim 1, wherein the bacteria is a gram positive or gram negative bacteria.

6. The vaccine of claim 1, wherein the bacteria is selected from the group consisting of Francisella tularensis, Burkholderia pseudomallei, and Yersinia pestis.

7. The vaccine of claim 1, wherein the virus is Influenza virus.

8. The vaccine of claim 1, wherein the antigen and the nucleic acid molecule are complexed to or within said liposome.

9. A vaccine for inducing an F. tularensis specific immune response in a subject, the vaccine comprising a complex comprising an effective amount of a cationic liposome, a non-coding nucleic acid molecule, and a F. tularensis membrane fraction in a pharmaceutically acceptable excipient.

10. The vaccine of claim 1, wherein the cationic liposome is octadecenolyoxy[ethyl-2-heptadecenyl-3 hydroxyethyl]imidazolinium chloride.

11. A method of immunizing a mammal comprising administering to said mammal a vaccine comprising an effective amount of a liposome, a non-coding nucleic acid molecule, and a bacterial antigen in a pharmaceutically acceptable excipient.

12. A method of treating or preventing a pathogen infection in a mammal in need thereof comprising administering to the mammal an effective amount of a composition comprising a liposome, a non-coding DNA, and a bacterial antigen in a pharmaceutically acceptable excipient,
or
a method of preventing a pathogen infection in an infected subject, the method comprising administering an effective amount of a composition comprising a cationic liposome, a non-coding nucleic acid molecule, and a pathogen antigen.

13-17. (canceled)

18. A method for inducing an F. tularensis specific immune response, the method comprising administering to a mammal an effective amount of a composition comprising cationic liposome, a non-coding nucleic acid molecule, and a F. tularensis membrane fraction in a pharmaceutically acceptable excipient.

19. The method of claim 1, wherein the composition is administered intraperitoneally (IP), subcutaneously (SC), intravenously (IV) or intranasally.

20. The method of claim 1, wherein the composition induced production of proinflammatory cytokines from antigen presenting cells.

21. The method of claim 1, wherein the composition reduces intracellular pathogen replication.

22. The method of claim 1, wherein the composition increases survival of the mammal following pathogen exposure compared to an untreated control mammal.

23. The method of claim 10, wherein the composition generates pathogen specific IgM in the mammal.

24. The method of claim 10, wherein the method prevents or treats a pathogen infection in the mammal.

25. The method of claim 24, wherein the pathogen infection is an infectious disease selected from the group consisting of bacterial meningitis, tularemia, influenza, plague, mellidosis, and pneumonicoccal mediated disease.

26. The method of claim 10, wherein the method inhibits intracellular growth of a pathogen.

27-32. (canceled)

33. A kit for preventing or treating a pathogen infection, the kit comprising an effective amount of a cationic liposome, a non-coding nucleic acid molecule, and a pathogen antigen.

34-36. (canceled)
